(19) United States
(12) Patent Application Publication
(10) Pub. No.: US 2015/0351784 A1
(43) Pub. Date: Dec. 10, 2015

(54) DECOMPRESSION INSTRUMENTS AND RELATED METHODS

(71) Applicant: Huy Dao, Turlock, CA (US)

(72) Inventor: Huy Dao, Turlock, CA (US)

(21) Appl. No.: 14/733,807

(22) Filed: Jun. 8, 2015

Related U.S. Application Data
(60) Provisional application No. 62/009,740, filed on Jun. 9, 2014.

(51) Int. Cl. A61B 17/22; A61B 17/285; A61B 17/28

(52) U.S. Cl. CPC A61B 17/22031; A61B 17/282; A61B 17/2841; A61B 17/285; A61B 2017/22037

(57) ABSTRACT

A decompression instrument is disclosed for removing impacted stool from a patient with long standing chronic constipation. The instrument is configured to enter the gastrointestinal tract through the anus to remove the impacted stool. The removed waste may be temporarily collected in a collection bag having a rim with adhesive for attaching to the patient during a decompression procedure.

### FIELD OF ART

The present invention is generally directed to medical instruments for non-operative decompression of impacted stool in the lower colon and related methods.

### BACKGROUND

Long standing chronic constipation can create serious obstruction to the bowel that may require emergency medical intervention. The problem can present over a span of a few days and can recur. The symptoms can include lower abdominal pain, gross abdominal distension, and vomiting. Shock and an elevation of temperature may be present if perforation to the gastrointestinal tract has occurred.

Any delay in treatment increases the risk of bowel ischaemia, perforation and fecal peritonitis. The majority of patients can be treated with non-operative decompression with elective surgery available for those so inclined.

### SUMMARY

A decompression instrument for removing impacted stool is disclosed. In an example, the device comprises a first extension comprising a collection head pivotably coupled to a second extension comprising a collection head, the two collection heads each comprising a perimeter defining an edge that contact one another along a parting line; the two collection heads defining a scoop end having a hollow interior; and wherein the two extensions each comprising a handle for use to manipulate the scoop end.

A decompression instrument for removing impacted stool in accordance with an alternative embodiment is disclosed. In an example, the alternative embodiment comprises a first body disposed around a second body; wherein said first body comprises an elongated body comprising an open scoop end having an inward lip and an open push end; wherein said second body comprises a spiral shaped body comprising a nose end and a shank end; and wherein the nose end and the shank is secured directly or indirectly to the first body.

Methods of making decompression instruments are understood to be within the scope of the present disclosure.

Methods of using decompression instruments are understood to be within the scope of the present disclosure.

Aspects of the present disclosure further comprise a decompression instrument for removing impacted stool comprising a first extension comprising a body having a first width, a recessed body section having a second width, which is less than the first width, and a collection head pivotably coupled to a second extension comprising a body having a first width, a recessed body section having a second width, which is less than the first width, and a collection head, the two collection heads each comprising a perimeter defining an edge that contact one another along a parting line; the two collection heads defining a scoop end having a hollow interior; and wherein the two extensions each comprising a handle at an end opposite the collection heads for use to manipulate the scoop end.

The decompression instrument can further comprise a perimeter defining an end opening at the scoop end, and wherein the parting line bisects the perimeter with the end opening.

The decompression instrument wherein the handle of the first extension can comprise a grip ring.

The decompression instrument wherein the edge at each collection head can comprise undulating surfaces.

The decompression instrument wherein the grip ring can have a gap.

The decompression instrument has a length and wherein the first extension can have a pin and the second extension can have a bore.

The decompression instrument wherein the pin and the bore can be located closer to a furthest most point at the scoop end than a furthest most point at the two handles and wherein the length of the decompression instrument is measured at the two furthest most points.

An aspect of the present disclosure ⁻further includes a method of performing a non-operative decompression of impacted stool comprising the steps: inserting a decompression instrument comprising a scoop end with a hollow cavity into an anus of a patient; activating the scoop end by translating, rotating, or both to the decompression instrument to collect impacted stool inside the hollow cavity; and removing the decompression instrument from the patient.

The method wherein the decompression instrument can be spread apart at two handles during the activating step.

The method can further comprise placing a collection bag near the anus to collect the impacted stool

The method wherein the collection bag can comprise adhesive and the adhesive is in contact with the patient.

The method wherein the collection bag can include a reinforced opening.

The method wherein the scoop end can comprise two collection heads that contact one another along a parting line.

The method can further comprise a perimeter defining an opening at the scoop end, and wherein the perimeter can be bisected by the parting line.

The method where the decompression instrument can comprise a first body having the scoop end and a second body located inside the first body; and wherein the second body can comprise a spiral shaped body.

The method wherein the scoop end can comprise a cutting edge.

The method can further comprise the steps: spreading two collection heads on the decompression instrument apart about a parting line; and moving the two collection heads toward one another.

A still further aspect of the present disclosure includes a decompression instrument for removing impacted stool comprising a first body disposed around a second body; wherein said first body comprises an elongated body comprising a scoop end having an inward lip and an open push end; wherein said second body comprises a spiral shaped body comprising a nose end and a shank end; and wherein the nose end and the shank is secured directly or indirectly to the first body.

The decompression instrument wherein the second body can include a cutting edge.

### BRIEF DESCRIPTION OF THE DRAWINGS

These and other features and advantages of the present devices, systems, and methods will become appreciated as the same becomes better understood with reference to the specification, claims and appended drawings wherein:

FIG. 1 is a side view of a decompression instrument provided in accordance with aspects of the present disclosure.

FIG. 2 is top view of the device of FIG. 1.

FIG. 3 is a perspective view of the device of FIG. 1.

FIG. 4 is a perspective view of the device of FIG. 1 with the handle manipulated to open the scoop end.

FIG. 5 is a perspective view of the device of FIG. 1 with the first extension spaced from the second extension.

FIG. 6 is a schematic view of a decompression procedure in which the device of FIG. 1 is used on a patient.

FIG. 7 is a schematic perspective view of an alternative decompression instrument provided in accordance with aspects of the present disclosure.

### DETAILED DESCRIPTION

The detailed description set forth below in connection with the appended drawings is intended as a description of the presently preferred embodiments of decompression instruments and related methods provided in accordance with aspects of the present disclosure and are not intended to represent the only forms in which the present devices, systems, and methods may be constructed or utilized. The description sets forth the features and the steps for constructing and using the embodiments of the present devices, systems, and methods in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features.

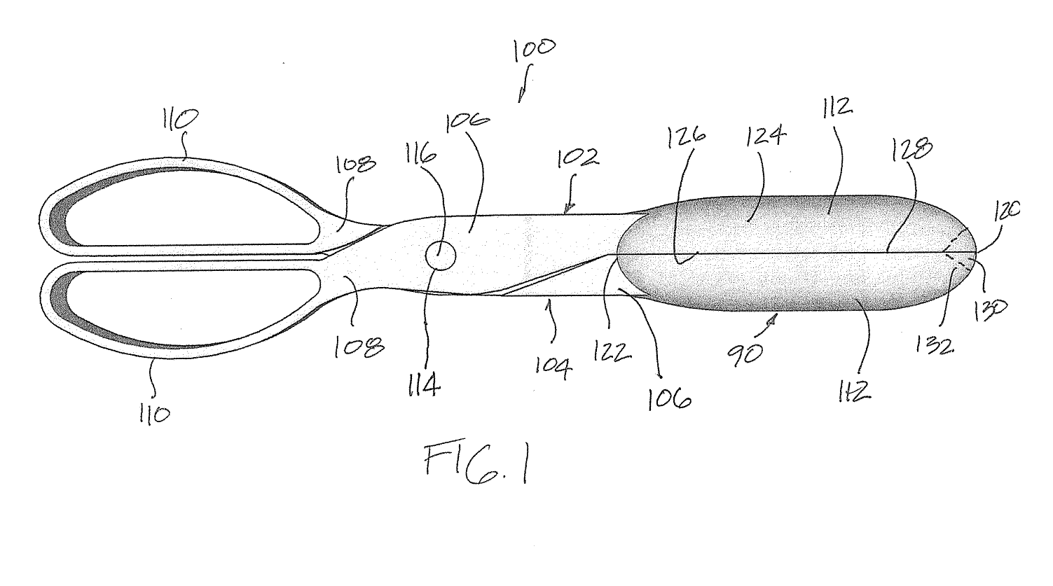

With reference now to FIG. 1, a side view of a decompression instrument or device 100 provided in accordance with aspects of the present disclosure is shown. The instrument 100 comprises a first extension 102 attached to a second extension 104. Each extension 102, 104 comprises a body 106, a handle 108 comprising a grip ring 110, and a collection head 112. As shown, the first extension 102 has a bore 114 and the second extension 104 has a pin 116 positioned in the bore 114 to pivotably couple the two extensions together. The attachment can be a close fit to a loose fit to enable quick connection and disconnection. The bore 114 and the pin 116 can reverse on the two extension bodies 106. When connected, the first and second extensions 102, 104 can pivot relative to one another about the pin 116, similar to a common hand-held scissor. Although the two grip rings 110 are shown with closed loops, the loops can be open, such as having a gap. The two grip rings 100 can be identical or similar or noticeably different, such as one with a closed loop and the other has a gap. One grip ring can also be round and the other generally elongated.

The device 100 has a length measured from the tip of the two collection heads 112 to the two grip rings 110. The two collection heads 112 together define a scoop end 90, which has a diameter and a length. In one example, the bore 114 and the pin 116 are positioned at about a half-way point of the length of the device 100. In another example, the bore 114 and pin 116 locations are located off-center at a point closer to the two collection heads 112 than the two grip rings 110. This allows for finer movement at the collection heads 112 when the two grip rings 110 at the handle 108 are manipulated, as further discussed below. In other examples, the location of the bore 114 and the pin 116 can be closer to the grip rings 110 than the scoop end 90. As shown, the bore 114 and the pin 116 are located off-center at a point closer to the two grip rings 110 than the two collection heads 112.

The device 100 may be made from reusable medical grade metals, such as stainless steel and the likes, which can be cleaned and undergo sterilization for re-use. In an alternative embodiment, the device 100 is made from a durable rigid plastic, such as from a PEEK material, polycarbonate, and other sufficiently rigid plastics. The device can be made for one time use as well as for re-use, such as from a material that conducive to cleaning and sterilizing. The pin and the bore can be integrally formed with the respective body 106 or subsequently formed from the respective body.

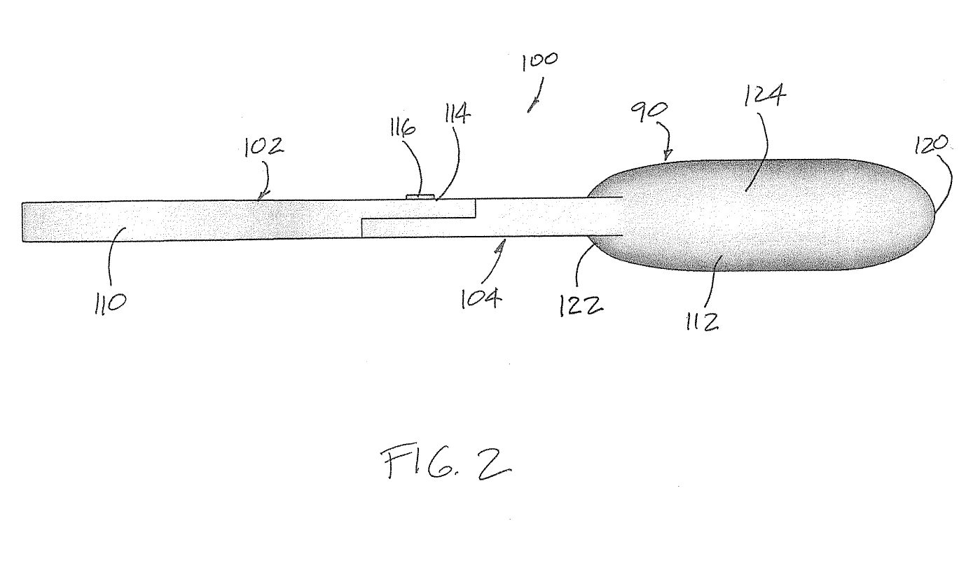

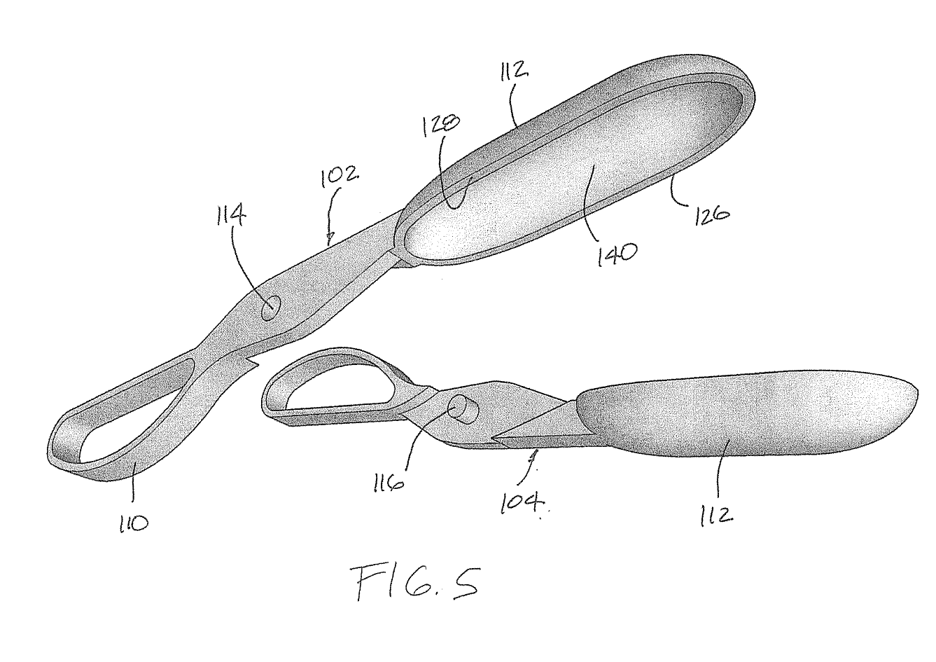

With reference now to FIG. 2, a top view of the device 100 of FIG. 1 is shown. The top view of FIG. 2 and the side view of FIG. 1 show the two collection heads 112 together having an elongated prolate spheroid shape. Another description of the two collection heads 112 is an elongated football shape or rugby ball shape. Of course, each collection head 112 is divided in half along the length of the elongated football shape, i.e., along the two tapered ends, such as along a first end 120 and a second end 122. Thus, each collection head 112 has a contoured outer body 124 and a perimeter 126 (FIG. 5) defining a contact edge 128. As shown, the two contact edges 128 of the two collection heads 112 contact one another along the entire perimeter 126 and the two have a parting line. In an alternative embodiment, the tip 130 of each collection head 112 is truncated along a line 132 to form a small distal opening at the tip of the device 100. The distal open can allow the device 100 to penetrate the obstructed gastrointestinal tract more easily. In some examples, the contact edges 128 are each continuous and smooth. In other examples, the contact edges are not smooth and not continuous. For example, the contact edge can be undulating or saw tooth in shape and arranged so that the saw tooth pattern of the two collection heads 126 mate or match.

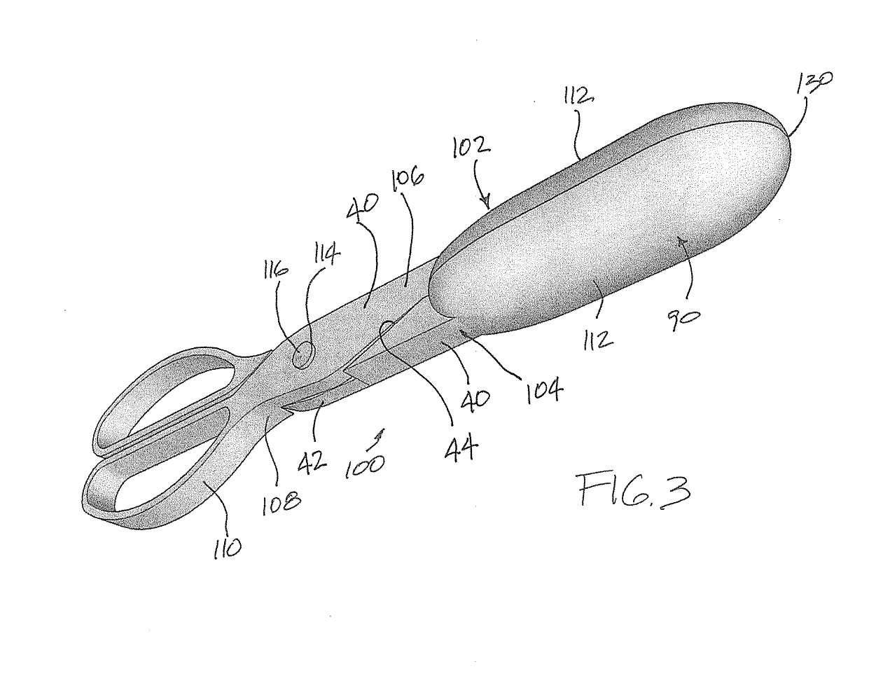

FIG. 3 is a perspective view of the device 100 of FIG. 1. In one example, the diameter of the scoop end 90 is about 1 inch to about 2 inches with 1.5 inches being more preferred. The length of the scoop end 90 is about 2.5 inches to about 6.5 inches with about 4.75 inches being more preferred. The overall length of the device 100 from the scoop end 90 to the grip rings 110 is about 10 inches to about 16 inches with about 12 inches being more preferred. In an example, an extended body section 40 of each body 106 has a first thickness and a recessed body section area 42 with a second thickness, which is less than the first thickness. Thus allows the two recessed body sections 42 of the two extensions 102, 104 to mate to form an overall thickness that is about the same as the first thickness. Additionally, the extending body section 40 of the two extensions 102, 104 each comprises an angled edge 44 to enable collapsing or closing of the two extensions 102, 104 at the extended body sections 40 when the scoop end 90 closes, as shown in FIG. 3.

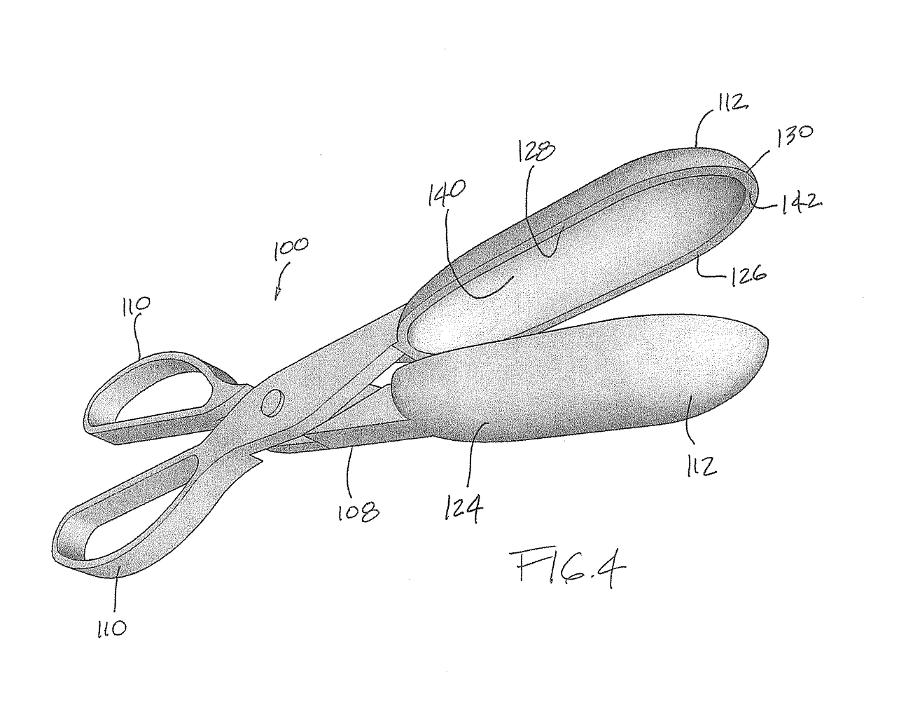

FIG. 4 is a perspective view of the device 100 of FIG. 1 with the grip rings 110 manipulated to open the scoop end 90. For example, a user can place a thumb through one of the grip rings 110 and one or more fingers in the other grip ring 110 and spread the fingers and the thumb to open the scoop end 90, similar to using a scissor. In the perspective shown, each collection head 112 has a body 124 with a hollow interior 140, a perimeter 126, and an edge 128. In one example, the edge at the tip 130, called a tip edge 142, is thinner than the remaining sections of the edge 128. This allows the two collection heads 112 to cut into the impacted stool during the non-operative decompression procedure, as further discussed below. As shown, the edge width is generally constant around the perimeter 126. For an embodiment with the truncated tip to form a small distal opening, the recessed area or cut back area forming the opening can also be provided with a relatively thinner edge to facilitate cutting into the impacted stool during the non-operative decompression procedure. In some examples, the scoop end resembles an elongated cylinder with an open distal end. A cutting mechanism is provided at the open distal end for cutting into the impacted stool during the non-operative decompression procedure. For example, the two handles can be connected to a rod that is connected to a cam or gearing mechanism to rotate a cutting tool at the scoop end. In other examples, the scoop end has a tapered elongated cylinder with the distal most end with the opening being smaller in diameter than a base of the tapered cylinder.

FIG. 5 is a perspective view of the first extension 102 and the second extension 104 separated from one another, wherein the pin 116 is not disposed in the bore 114. The device 100 may be separated as shown in FIG. 5 for cleaning and sterilization purposes following a decompression procedure. In alternative embodiment, the device 100 is disposed of following use pursuant to established procedures.

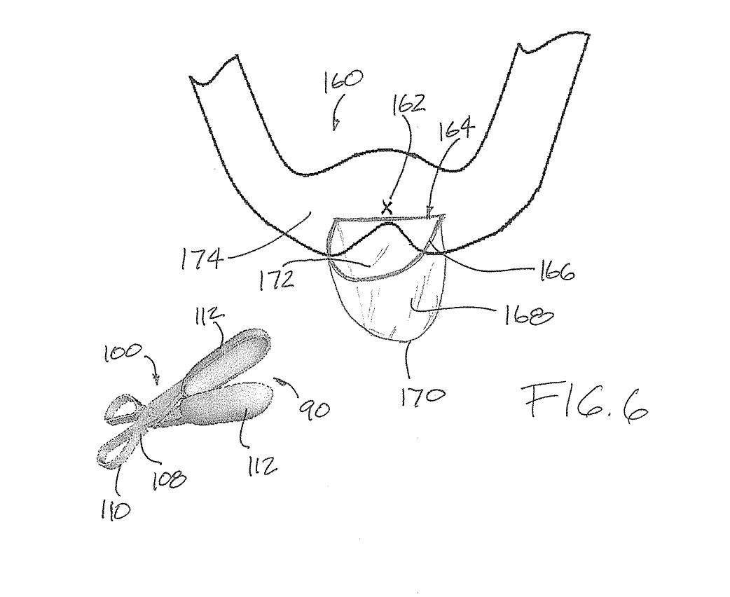

FIG. 6 is a schematic view of a patient 160 undergoing a non-operative decompression procedure. As shown, the patient is laying 160 in a supine position with the face up. The anus 162 is exposed for the decompression procedure. In one example, a collection bag 164 having a rim 166 and a bag body 168 is shown attached to the patient for collecting impacted stool as the stool is removed from the colon. The bag body 168 has a closed end 170 and an open end 172 in communication with the rim 166. The rim 166 is provided with an adhesive, such as repositionable tape or pressure-sensitive tape (PSA). The rim 166 is adhered to the patient, such as to the buttock 174, just below the anus 162. The rim 166 can be reinforced so that the open end 172 can spread wide to collect the impacted stool as it is being removed from the anus. For example, the rim can be reinforced with a wire or other rigid bodies, such as a non-metallic rod. The bag can be made from a thermoplastic material and can be clear, opaque, or semi-opaque. A cap or lid can be incorporated with the bag so that following use, the cap or lid can close over the open end 172. For example, an extended flap can be provided with adhesive. The extended flap can close over the open end following use and the adhesive can adhere to the bag body to close off the open end 172.

With further reference to FIG. 6, the decompression instrument or device 100 can be on the patient 160 with or without the collection bag 164. For example, a tray or other prep sheets or materials may be placed below the patient during the non-operative procedure to collect the impacted stool. In one example, the scoop end 90 is first placed near the entrance of the anus 162 with the two collection heads 112 partially opened. The scoop end 90 is then inserted into the anus and the handle 108 is used to manipulate the two collection heads. As the practitioner slowly works his way into the anus and removes sections of impacted stool from the patient, the device can open to deposit the removed stool into the collection bag 164.

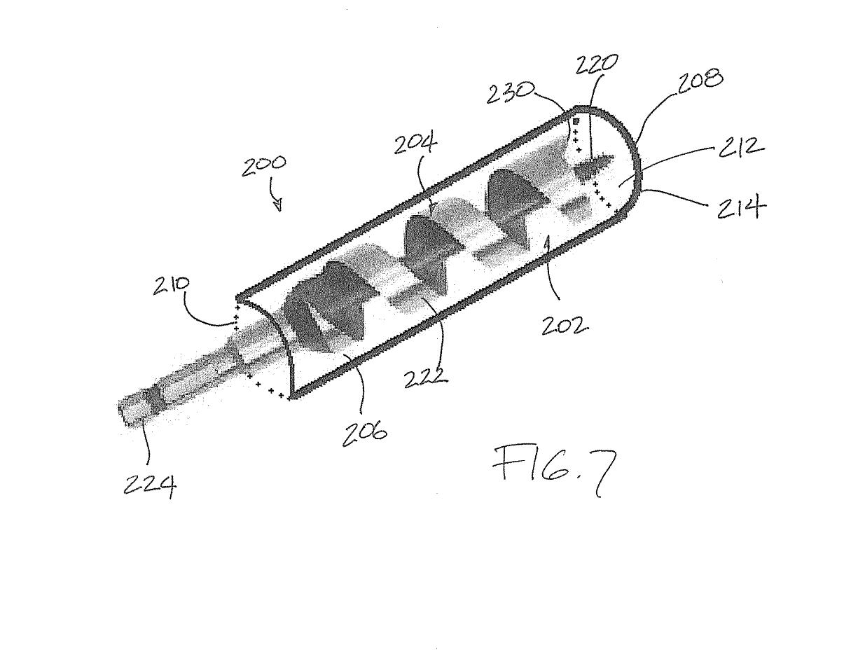

FIG. 7 is a schematic perspective view of an alternative decompression instrument or device 200 comprising a first body 202 surrounding a second body 204. As shown, the first body 202 is separately formed and placed over or around the second body 204. In an alternative example, the first body is fixedly secured to the second body 204 so that the device 200 moves, such as translate, rotate, or both, as a single unit. As shown, the first body 202 comprises an elongated tube 206 comprising an outside diameter, a scoop end 208, and a push end 210. The scoop end 208 has an end opening 212 comprising a lip 214. In one example, the lip 214 is inwardly extending so that no sharp or abrupt edges are provided at the scoop end 208 that can cut into and possibly injure the gastrointestinal tract during a non-operative decompression procedure. In an example, the first body 202 is an elongated cylinder with two open ends. The first body 202 can be made from a plastic material or from a metal.

The second body 204 comprises a nose section 220, a spiral shaped body 222, also call a spur or twist, and a shank 224. In one example, the nose 220 and the shank 224 of the second body are held by a support structure, such as a bearing or a sleeve, to rotatably secure the second body 204 to the first body 202. In other words, the device 200 is configured translate with or without rotation into the gastrointestinal tract by pushing alone or by pushing with rotation of the first body 202 at the push end 210. As the first body is advanced slowly into the gastrointestinal tract, the second body 204 is slowly rotated at the shank end 224 using a handle (not shown) or a rotatable instrument, which may be manually rotatable or motorized.

The second body 204 has a leading edge 230 designed to advance into the impacted stool as the device 200 is advanced into the anus during the decompression procedure. In an example, the leading edge is made with a sufficiently sharp cutting edge 230 to enable cutting into the impacted stool when the second body 204 is rotated as the device is being inserted into the anus. The spiral body 222 is provided with wide lands to allow the impacted stool to move out the open push end 210 of the first body 202. The first body 202 preferably has an outside cross-sectional dimension of about ¾-inch to about 1¾ inches with about 1 inch to about 1¼ inches being more preferred.

The device of FIG. 7 may be used in a similar procedure as described above with reference to FIG. 6, such as with or without a collection bag. Thus, an aspect of the present disclosure is understood to include a device for use to perform a non-operative decompression procedure. The device comprises a scoop end that is insertable into the anus to remove impacted stool. The scoop end has a movable mechanism for removing the impacted stool, such as for cutting, chipping, grabbing, boring, or scooping. The movable mechanism can comprise a rotational movement, a pivoting movement, translation, or combinations thereof. The scoop end should have smooth contoured surfaces so as not to injure the gastrointestinal tract.

Although limited embodiments of decompression instruments or devices and their components have been specifically described and illustrated herein, many modifications and variations will be apparent to those skilled in the art. Accordingly, it is to be understood that the decompression instruments or devices and their components constructed according to principles of the disclosed device, system, and method may be embodied other than as specifically described herein. The disclosure is also defined in the following claims.

What is claimed is:

1. A decompression instrument for removing impacted stool comprising a first extension comprising a body having a first width, a recessed body section having a second width, which is less than the first width, and a collection head pivotably coupled to a second extension comprising a body having a first width, a recessed body section having a second width, which is less than the first width, and a collection head, the two collection heads each comprising a perimeter defining an edge that contact one another along a parting line; the two collection heads defining a scoop end having a hollow interior; and wherein the two extensions each comprising a handle at an end opposite the collection heads for use to manipulate the scoop end.

2. The decompression instrument of claim 1, further comprising a perimeter defining an end opening at the scoop end, wherein the parting line bisects perimeter with the end opening.

3. The decompression instrument of claim 1, wherein the handle of the first extension comprises a grip ring.

4. The decompression instrument of claim 1, wherein the edge at each collection head comprises undulating surfaces.

5. The decompression instrument of claim 3, wherein the grip ring has a gap.

6. The decompression instrument of claim 1, wherein the decompression instrument has a length and wherein the first extension has a pin and the second extension has a bore.

7. The decompression instrument of claim 6, wherein the pin and the bore are located closer to a furthest most point at the scoop end than a furthest most point at the two handles and wherein the length of the decompression instrument is measured at the two furthest most points.

8. A method of performing a non-operative decompression of impacted stool comprising the steps:
inserting a decompression instrument comprising a scoop end with a hollow cavity into an anus of a patient;
activating the scoop end by translating, rotating, or both to the decompression instrument to collect impacted stool inside the hollow cavity; and
removing the decompression instrument from the patient.

9. The method of claim 8, wherein the decompression instrument is spread apart at two handles during the activating step.

10. The method of claim 8, further comprising placing a collection bag near the anus to collect the impacted stool

11. The method of claim 8, wherein the collection bag comprises adhesive and the adhesive is in contact with the patient.

12. The method of claim 8, wherein the collection bag has a reinforced opening.

13. The method of claim 8, wherein the scoop end comprises two collection heads that contact one another along a parting line.

14. The method of claim 13, further comprising a perimeter defining an opening at the scoop end, and wherein the perimeter is bisected by the parting line.

15. The method of claim 8, where the decompression instrument comprising a first body having the scoop end and a second body located inside the first body; and wherein the second body comprises a spiral shaped body.

16. The method of claim 8, wherein the scoop end comprises a cutting edge.

17. The method of claim 8, further comprising:
spreading two collection heads on the decompression instrument apart about a parting line; and
moving the two collection heads toward one another.

18. The method of claim 12, wherein the collection bag comprises a cover for covering the reinforced opening.

19. A decompression instrument for removing impacted stool comprising a first body disposed around a second body;
wherein said first body comprises an elongated body comprising a scoop end having an inward lip and an open push end;
wherein said second body comprises a spiral shaped body comprising a nose end and a shank end; and
wherein the nose end and the shank is secured directly or indirectly to the first body.

20. The decompression instrument of claim 18, wherein the second body has a cutting edge.
